(19) United States
(12) Patent Application Publication
(10) Pub. No.: US 2014/0163306 A1
(43) Pub. Date: Jun. 12, 2014

(54) MAGNETIC DEVICE WITH REPULSIVE MAGNETIC ELEMENTS

(71) Applicant: Ronald Travis Sykes, Tallahassee, FL (US)

(72) Inventor: Ronald Travis Sykes, Tallahassee, FL (US)

(21) Appl. No.: 13/926,995

(22) Filed: Jun. 25, 2013

Related U.S. Application Data
(60) Provisional application No. 61/690,374, filed on Jun. 25, 2012.

(51) Int. Cl. A61N 2/06; A44C 25/00

(52) U.S. Cl. CPC A61N 2/06; A44C 25/00
USPC 600/15; 63/1.11; 335/306

(57) ABSTRACT

A magnetic device is disclosed for entertainment, education or magnetic therapy. The device includes a transparent annular tube having magnetic elements that slide within the tube cavity. The magnetic elements are arranged within the tube whereby the same poles on the magnetic elements create repulsive forces between them as the elements slide within the tube cavity, and contact between each magnetic element is prevented and the magnetic elements remain spatially separated within the tube. The magnetic device includes a wearable structure holding the tube with the magnetic elements.

### PRIORITY CLAIM

This application claims benefit of U.S. Provisional Application No. 61/690,374 filed by Ronald Travis Sykes on Jun. 25, 2012 and entitled U-Balanced.

### BACKGROUND OF THE INVENTION

1. Field of the Invention

The present invention relates to novelty items and therapeutic devices and, more particularly, relates to magnetic therapy devices that may be wearable such as jewelry, biomedical aids, amusement devices, and other like wearable devices utilizing magnets and magnetism.

2. Review of the Prior Art

Wearable magnetic devices as biomedical aids have long been believed to have potential therapeutic benefits. Interest in magnet therapy for medical conditions continues because there is insufficient information to know how magnets might work in the body to treat pain or disease. There are a variety of electromagnetic fields that naturally occur within the body. For example, nervous system transmissions and related muscle contractions are associated with magnetic activity, and the heart generates a large magnetic field in the body. Studies have indicated that magnetic therapy may help with chronic conditions such as arthritis, chronic pain, and fibromyalgia. Therefore, magnets may be worn to interact with the natural magnetic fields in the body and provide actual or perceived relief. However, the human body includes diverse members. Magnets also fascinate and amuse because of the attractive and repulsive properties of magnetic forces. Therefore, magnets may be provided in novel configurations for entertainment and education. By providing effective and flexible structural elements, this interest in wearable magnets for therapy and amusement can be met for a variety of positions on the body in accordance with a person's desires.

### SUMMARY OF THE INVENTION

This invention provides a wearable magnetic device with potential therapeutic qualities in the nature of a bracelet, watch, necklace, brace device, or ring that includes an annular tube having two or more magnetic elements movably disposed within the tube. The magnetic elements are disposed within the tube so that the magnetic poles of movable magnetic elements (or elements) face like magnetic poles of the paired complementary element. The annular tube (or tube) includes an elongated cavity, preferably visible through the tube wall, and the movable magnetic elements slide within the cavity and each element repels from the paired complementary element when moved within the repelling range of the magnetic force of the like pole of the complementary element.

It is therefore an object of the invention to provide a magnetic novelty item.

It is another object of this invention to provide a therapeutic magnetic device having moving magnetic elements with at least two or more magnets inside of an annular tube in the form of a ring.

It is another object of this invention to provide a magnetic device having an annular tube in the form of a ring that is hollowed out in the center forming an elongated cavity in at least one magnetic element can travel freely inside of the cavity subject to repulsive magnetic forces between magnetic elements.

It is another object of this invention to provide a magnetic device having an annular tube comprising magnetic elements that is manufactured through insertion of magnetic elements between portions of an annular tube that are adhered together to seal the magnetic elements therein.

It is another object of this invention to provide a therapeutic magnetic device that can be adapted to be worn as a bracelet, watch, necklace, brace device or ring on the human body.

It is still another object of this invention to provide a magnetic novelty item having an annular tube with internal cavity, at least two moving magnetic elements that slide within the cavity, and the magnetic elements arranged so that the like poles of the magnetic elements repel one another within a tube when the magnetic elements move within proximity of the other's repulsive magnetic force.

It is still another object to provide a magnetic device with an annular tube in the form of a ring that can be either circular or oval in shape.

It is still another object of this invention to provide entertainment and education through providing a transparent annular tube comprised of a clear ring made of glass, plastic or other material, and observation of magnetic elements within the annular tube repelling one another and being balanced and spatially separated through the magnetic repulsive force of the magnetic elements.

It is yet another object of this invention to provide a therapeutic magnetic device having an annular, curved or linear tube with an internal cavity shaped as a circular ring, oval ring, ring with tubular cross members, cross, or linear tube, and having at least two moving magnetic elements that slide within the cavity and being arranged so that the magnetic elements will magnetically repulse one another, and the magnetic device being arranged to be attached or worn on a person's fingers, wrist, neck, arm, knee, ankle, leg, chest or back.

It is yet another object of this invention to provide a magnetic device that can be attached to or worn by an animal.

These and other objects of the invention that will become apparent to one skilled in the art in view of the detailed description that follows. The invention resides in the novel construction, combination, and arrangement of parts substantially as hereinafter described, and more particularly defined in the claims, it being understood that changes to the precise embodiment of the invention disclosure herein are meant to be included within the scope of the claims.

### BRIEF DESCRIPTION OF THE DRAWINGS

FIG. 1 is a perspective view of a preferred first embodiment of the device with repulsive magnetic elements of this invention.

FIG. 2 is a perspective view of the device of FIG. 1 in a wearable pouch.

FIG. 3 is a plan view of the device of FIG. 1 with an elbow brace.

FIG. 4 is a plan view of the device of FIG. 1 with a back brace.

FIG. 5 is a plan view of the device of FIG. 1 with a patella brace.

FIG. 6 is a perspective view of the device of FIG. 1 with a wrist band.

FIG. 7 is a plan view of the device of FIG. 1 with a wrap having hook and loop fasteners for attachment to the human body.

FIG. 8 is a plan view of the device of FIG. 1 with a knee brace.

FIG. 9 is a plan view of the device of FIG. 1 with an elbow brace.

FIG. 10 is a plan view of the device of FIG. 1 with an ankle brace.

FIG. 11 is a plan view of the device of FIG. 1 with a watch band.

FIG. 12 is a plan view of the device of FIG. 1 attached to a necklace.

FIG. 13 is a plan view of the device of FIG. 1 attached in plurality with annular rings to form a necklace.

FIG. 14 is a plan view of the device of FIG. 1 attached to a chain and pin with clasp.

FIG. 15 is a plan view of another embodiment of the device of this invention with repulsive magnetic elements.

FIG. 16A is a plan view of another embodiment of the device of this invention with repulsive magnetic elements.

FIG. 16B is a plan view of another embodiment of the device of this invention with repulsive magnetic elements.

FIG. 17A is a plan view of another embodiment of the device of this invention with repulsive magnetic elements.

FIG. 17B is a plan view of another embodiment of the device of this invention with repulsive magnetic elements.

FIG. 18A is a plan view of another embodiment of the device of this invention with repulsive magnetic elements.

FIG. 18B is a plan view of yet another embodiment of the device of this invention with repulsive magnetic elements.

### DETAILED DESCRIPTION OF THE INVENTION

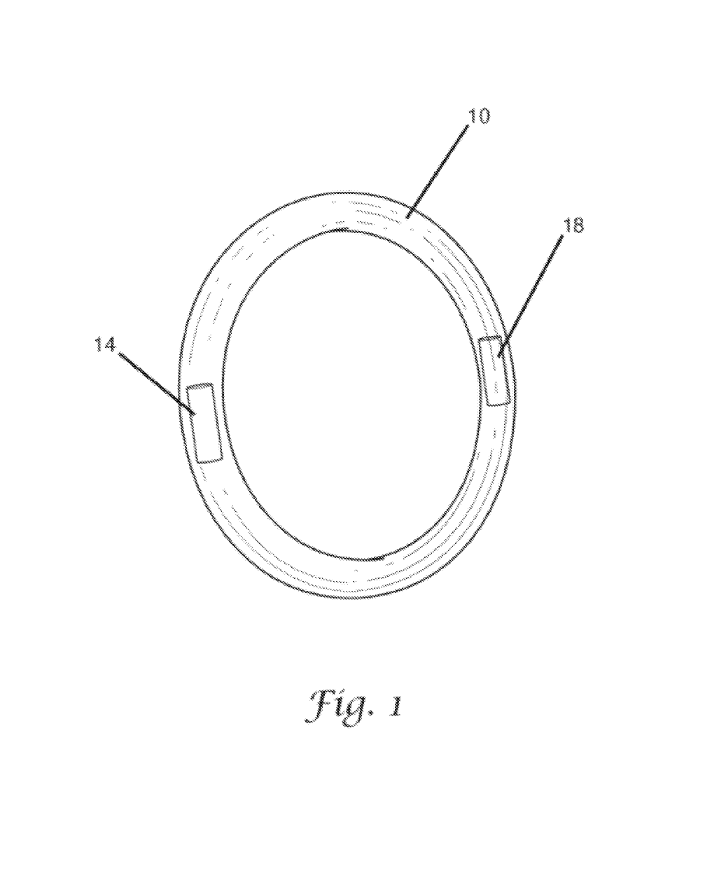

Referring now to the drawings, FIG. 1 shows an illustrative embodiment of the magnetic device of the invention. The magnetic device has a tube 10 that defines an elongated cavity for movement of repulsive magnetic elements therein. At least two first magnetic elements comprising a first magnetic element 14 and second magnetic element 18 are provided within the elongated cavity and slide within the cavity to provide movement that may be therapeutic, entertaining and educational. The magnetic elements each have a north pole and a south pole, i.e. opposing ends of the magnetic elements with magnetic poles. The ends of the magnetic elements comprising the same polarity create repulsive forces between each of the magnetic elements. When the elements approach one another within such proximity that the magnetic fields are of sufficient strength, the magnetic elements repulse one another. Thus, the elements slide within the elongate cavity of the tube and align and repulse one another as they slide closer together within the tube. Unless one uses a means to manipulate the magnetic elements within the tube with sufficient force, the repulsive force between the magnetic elements will prevent contact between the magnetic elements, and the magnetic elements will remain spatially separated within the elongated cavity.

As shown in the exemplary embodiment of FIG. 1 the tube 10 is an annular ring of circular or oval shape in which the magnetic elements 14, 18 can travel along the entire interior surface circumference defining the elongated cavity of the annular tube. The tube 10 is substantially transparent in the embodiment to provide for entertainment and education using the device, whereby the travel of the magnetic elements 14, 18 and spatial separation maintained by the repulsive magnetic forces can be observed.

The magnetic repulsion force of the magnetic elements may be specifically calibrated to improve the balance and separation of the elements within the elongated cavity of the tube. In one embodiment, the magnetic elements may include sufficient magnetic force to maintain a substantially equidistant spatial relationship of the magnetic elements within the tube. It is desirable that the magnetic elements slide and move easily within the elongated cavity of the tube. Therefore, the cavity of the tube may be engineered with a smooth friction reduce surface, and the magnetic elements or the interior surface of the tube may be lubricated with a lubricant or friction reducing treatment.

FIG. 2 illustrates an alternative embodiment of the magnetic device in which an annular tube 10 containing the magnetic elements 14, 18 is placed within a pouch 22. The pouch assists in holding and placing the tube of the device on the human body. In the example shown, the pouch includes a first side composed of fabric and a second side 26 including a hook and loop fastening material for attachment of the pouch to wraps or braces for use with the human body.

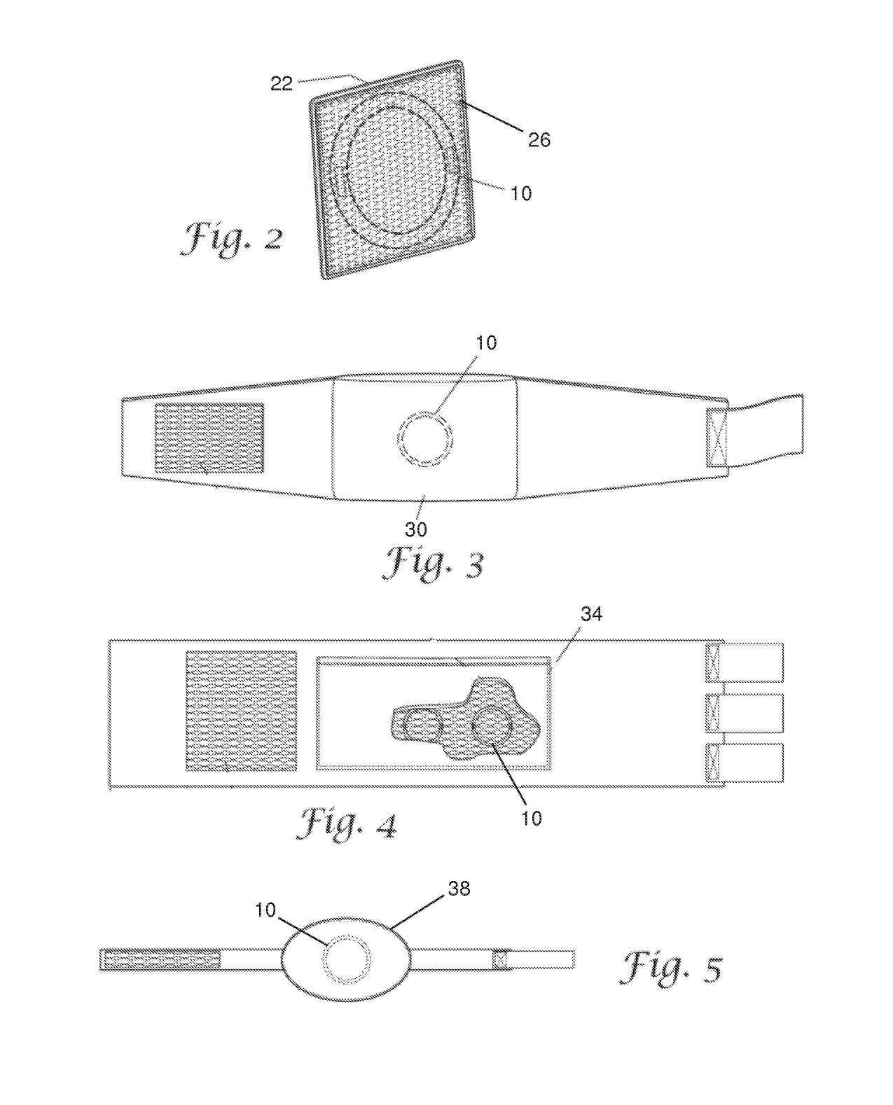

FIGS. 3 through 14 illustrate various embodiments of the magnetic device including a wearable structure for various therapy related to various parts of the human body. As shown in FIG. 3, the annular tube 10 containing the magnetic elements 14, 18 is embedded within a section of an elbow brace 30. The example elbow brace holds the tube about the center of the elbow brace within a padded pouch. The elbow brace may be attached using the pouch 22 and hook and loop fastener as shown in FIG. 2. The example elbow brace 30 includes soft and pliable material, usually attached by hook and loop fastening that is wrapped about a person's arm, and the portion of the elbow brace holding the tube is arranged onto the person's elbow for treatment.

In FIG. 4 the annular tube 10 is placed in a back brace 34 for treatment of a person's back. The back area is larger than the elbow area to be treated. Therefore, the area of the back brace 34 in which the tube is embedded may be larger and several tubes may be embedded to increase and enhance the therapeutic benefits of the magnetic device. Alternatively, the location of the annular tube 10 within the back brace 34 may be adjusted to situate the magnetic elements in the area of the person's back needing therapy. The back brace 34 and other pliable material braces include suitable means for attachment of the brace to a person's body such as hook and loop fastening via tabs and strips situated on the ends of the brace.

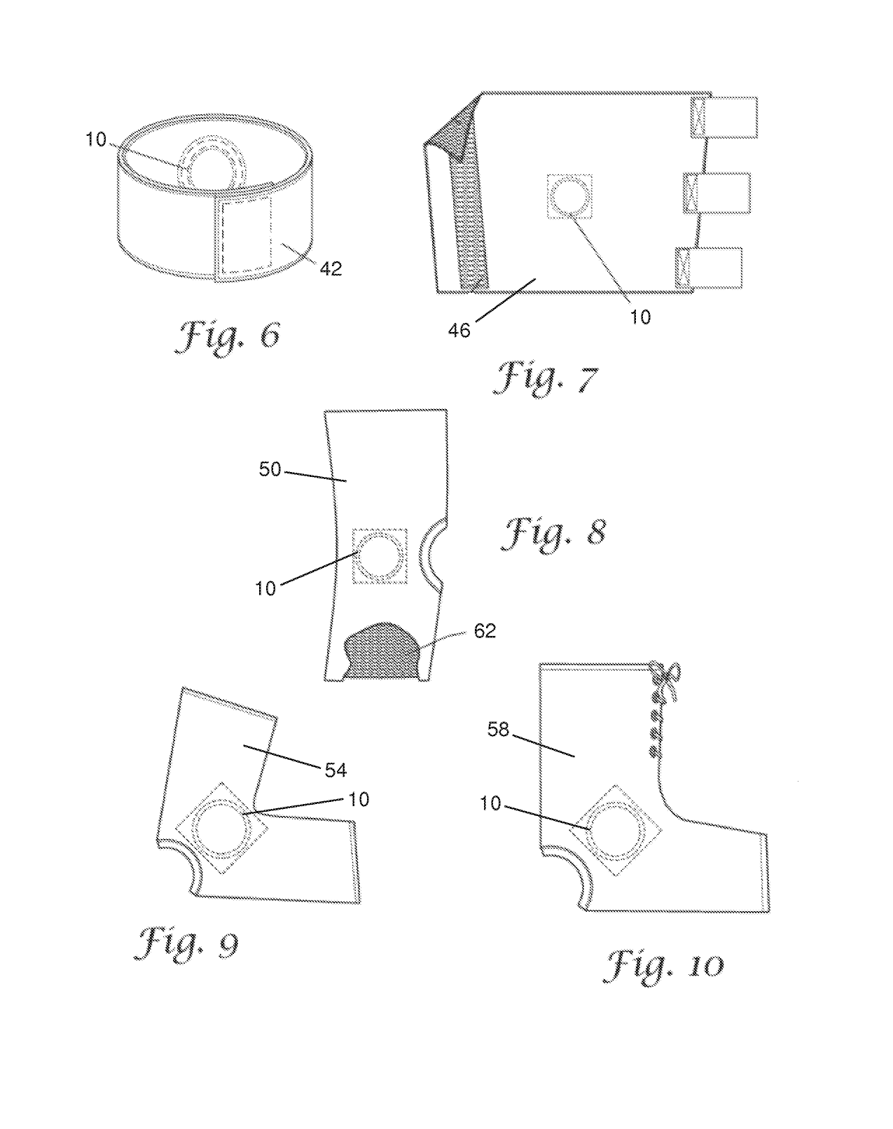

In additional embodiments, wraps are provided in the magnetic device for placement of the tube with magnetic elements on the body. FIG. 5 shows an annular tube 10 of the magnetic device attached to a patella brace 38. A pad may be provided on the brace 38 for attachment of the annular tube so that the tube is comfortably held against a person's body. FIG. 6 shows the annular tube 10 embedded within a wristband 42, and FIG. 7 shows the annular tube 10 embedded within a generic wrap 46 that may be used for the thigh, calf or arm. In each of these examples, hook and loop fastener is used for the wrap. The tube may be embedded, sewn in, or attached to a wrap using suitable means for positioning the tube on the affected body part.

FIGS. 8, 9 and 10 illustrate that sleeves are provided in the magnetic device for placement of the tube with magnetic elements on additional parts of the body. FIG. 8 shows an exemplary knee brace sleeve 50 with the annular tube 10 embedded in the sleeve for placement on the knee, making the device suitable for treatment of the knee. FIG. 9 illustrates the annular tube 10 embedded in an elbow sleeve brace 54, and FIG. 10 illustrates the annular tube embedded in an ankle brace sleeve 58. The annular tube may be embedded in an existing pouch of the sleeve or attached by suitable means to the sleeve, such as using a pouch 22 like that provided in FIG. 2. The surface of the sleeves may be provided with hook and loop fastener 62 for each attachment and removal of the annular tube. Thereby, the magnetic device may be modular for convenience.

In FIG. 11, the annular tube is attached to a watch band 66. As shown, the watch band may attach on each side to the annular tube 10. A transparent annular tube will allow the wearer to observe the magnetic elements 14, 18 within the annular tube while wearing the device on the wrist using the watch band. This configuration is convenient for treatment of the wrist and forearm and provides for easy display of the characteristics of the magnetic elements.

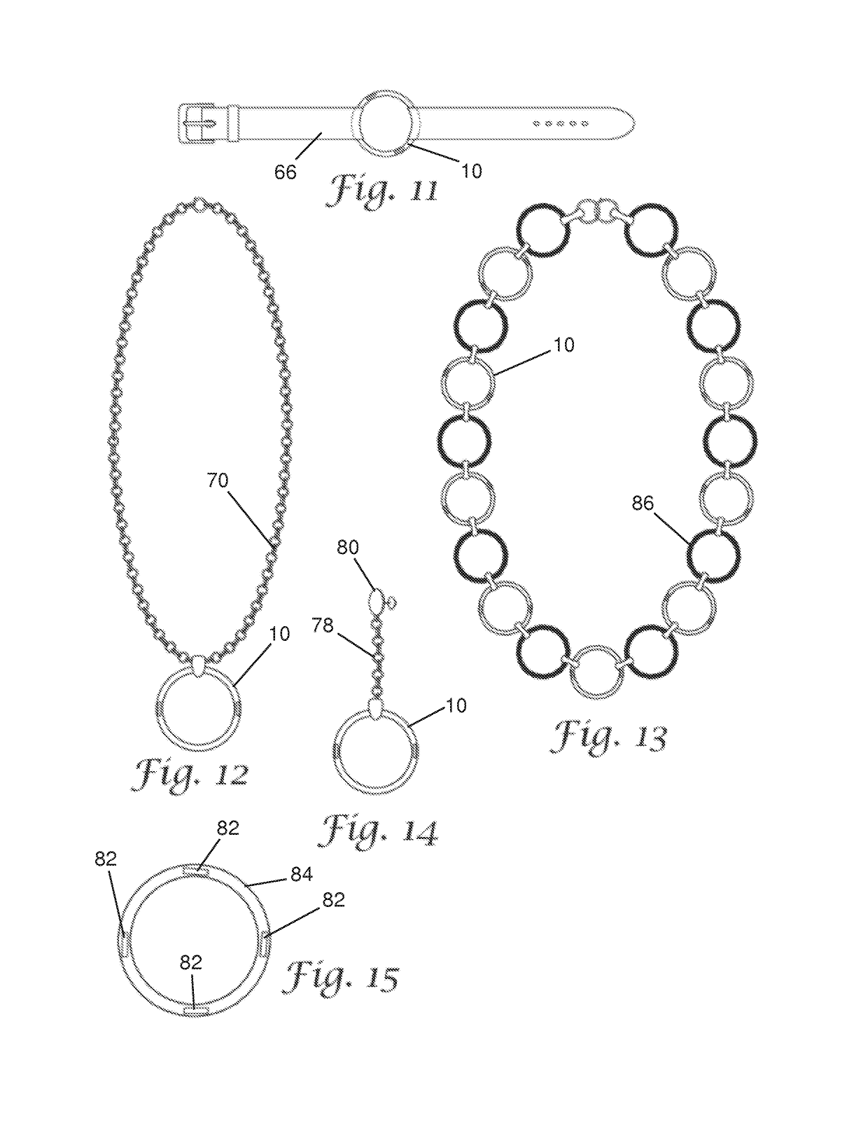

In FIG. 12, the annular tube 10 is attached as a pendant to a necklace 70 to form the magnetic device. The necklace 70 permits the annular tube 10 with magnetic elements 14, 18 to hang about the level of the lower neck or upper chest. In this arrangement, the device may be used for treatment of the neck and chest areas and may be readily visible for decorative entertainment and display of the characteristics of the magnetic elements. FIG. 13 includes a plurality of annular tubes 10 connected together and may connect in combination with decorative rings 74 or other necklace elements for form a necklace with several annular tubes and magnetic elements. This arrangement may have additional advantage for therapy related to neck pain and stiffness. FIG. 14 is similar to FIG. 12 but shows the annular tube 10 attached as a pendant to a chain 78 and pin 80 for use as an earring or decorative pendant elsewhere on the body.

The tube of the device may include more than two magnetic elements that repulse one another. For example, FIG. 15 illustrates an embodiment having four magnetic elements 82 within an annular tube 84. The additional third magnetic element and fourth magnetic element are each received in sliding relationship within the elongated cavity of the annular tube 84. The repulsive forces of like polarity of the opposing ends of the magnetic elements 82 maintain the four magnetic elements in spaced relationship within the annular tube.

The annular tube of the device may be sized to be worn as a bangle on a person's wrist or as a finger ring. The annular tube, like that shown in FIG. 15, may come in several sizes. The elongated channel of the tube may also be enlarged or reduced to hold different sized magnetic elements. Models sized accordingly and proportionately as bangles and rings may be made to be worn by men, women and children on their wrists, fingers or ankles.

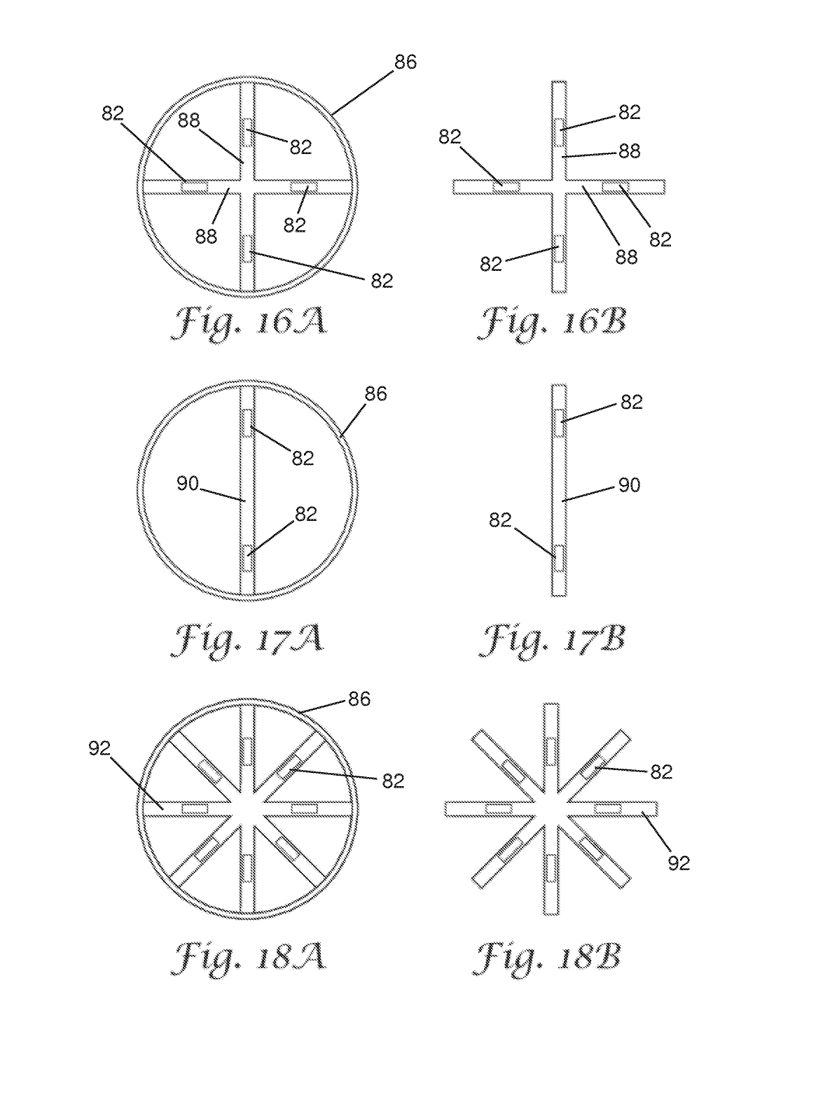

Alternative embodiments shown in FIGS. 16A, 16B, 17A, 17B, 18A and 18B show the magnetic elements 82 retained with the elongated cavity of the tubes having various configurations. In FIG. 16A an annular ring 86 includes cross-member tubes 88 in a cross-shaped configuration containing the magnetic elements 82. In FIG. 16B the cross-member tubes 88 are provided in cross-shaped configuration without the annular ring 86. In FIG. 17A an annular ring 86 includes a single linear cross-member tube 90 spanning the inner diameter of the annular ring and containing the magnetic elements 82. In FIG. 17B a single linear tube 90 is provided without the annular ring 86. In FIG. 18A the annular ring 86 includes multiple cross-member tubes 92 in a star-shaped configuration containing the magnetic elements 82. In FIG. 18B the multiple cross-member tubes 92 are provided in star-shaped configuration without the annular ring 86. In each of these configurations, the magnetic poles of the magnetic elements 82 are arranged with opposing like poles to repulse one another within the individual tubes. These tubes are linear and permit sliding movement of the magnetic elements from one end to the next. Because of the repulsive forces, the magnetic elements will remain spaced apart within these tubes.

What is claimed is:

1. A magnetic device comprising:
a tube having an elongated cavity;
a first magnetic element received in sliding relationship within the elongated cavity;
a second magnetic element received in sliding relationship within the elongated cavity; and
said first and second magnetic elements having a north pole and a south pole and received in said elongated cavity in such a way that poles of the magnetic elements comprising the same polarity create repulsive forces between each of the magnetic elements when the elements slide and approach one another within the elongated cavity, wherein contact between each magnetic element is prevented and the magnetic elements remain spatially separated within the elongated cavity.

2. The magnetic device of claim 1 wherein said tube is substantially transparent.

3. The magnetic device of claim 1 wherein said tube is annular and said elongated cavity includes an annular interior surface defining the elongated cavity.

4. The magnetic device of claim 3 wherein said magnetic elements slide about the entire annular interior surface of the elongated cavity.

5. The magnetic device of claim 4 wherein the repulsive forces between each of the magnetic elements are calibrated to maintain the magnetic elements within the elongated cavity in a substantially equidistant spatial relationship within the elongated cavity.

6. The magnetic device of claim 3 wherein said annular interior surface include a lubricant or friction reducing treatment.

7. The magnetic device of claim 4 wherein a third magnetic element is received in sliding relationship within the elongated cavity and a fourth magnetic element is received in sliding relationship within the elongated cavity.

8. The magnetic device of claim 1 in which said tube is received within a pouch including at least one side with a hook and loop fastener.

9. The magnetic device of claim 1 in which said tube is received by an elbow brace.

10. The magnetic device of claim 1 in which said tube is received by a back brace.

11. The magnetic device of claim 1 in which said tube is received by a patella brace.

12. The magnetic device of claim 1 in which said tube is received by a wristband.

13. The magnetic device of claim 1 in which said tube is received by a wrap having hook and loop fasteners for attachment of the wrap to a human body.

14. The magnetic device of claim 1 in which said tube is received by a knee brace sleeve.

15. The magnetic device of claim 1 in which said tube is received by an elbow brace sleeve.

16. The magnetic device of claim 1 in which said tube is received by an ankle brace sleeve.

17. The magnetic device of claim 3 in which said tube is attached to a watch band.

18. The magnetic device of claim 3 in which said tube is attached to a necklace as a pendant feature of the necklace.

19. The magnetic device of claim 3 including a plurality of the tubes connected in combination with a plurality of decorative rings to form a necklace.

20. The magnetic device of claim 3 in which said tube is attached to a chain as a pendant and an opposite end of the chain includes a pin and clasp.

21. The magnetic device of claim 1 including an annular ring and wherein said tube forms cross-members of the annular ring.
